(19) United States
(12) Patent Application Publication
(10) Pub. No.: US 2024/0382166 A1
(43) Pub. Date: Nov. 21, 2024

(54) CHARGED PARTICLE IMAGING SYSTEM AND USE THEREOF

(71) Applicant: Rodney HERRING, Victoria (CA)

(72) Inventor: Rodney HERRING, Victoria (CA)

(21) Appl. No.: 18/555,416

(22) PCT Filed: Apr. 20, 2022
(86) PCT No.: PCT/CA22/50605

§ 371 Date: Oct. 13, 2023

(30) Foreign Application Priority Data
Apr. 20, 2021 (CA) 3115634

(51) Int. Cl. A61B 6/40; A61B 6/00; A61B 6/42; G21K 1/087; H01J 37/147

(52) U.S. Cl. CPC A61B 6/405; A61B 6/4035; A61B 6/4258; A61B 6/482; G21K 1/087; H01J 37/1472; H01J 2237/244; H01J 2237/24564

(57) ABSTRACT

A method of imaging an object in a first material having a different charge density to the object is provided, the method comprising: focusing a charged particle beam to a virtual charged particle beam source in the first material; moving the virtual charged particle beam source in and around the object to provide at least one charged particle reflected object beam or at least one charged particle refracted object beam and at least one charged particle bypass beam, wherein the charged particle reflected object beam or the charged particle refracted object beam and the charged particle bypass beam intercept one another to form an interference zone; and defocusing the interference zone to provide a Fresnel fringe, the Fresnel fringe forming an image of the object; or focusing the virtual charged particle beam source on the object to provide a first lower energy charged particle beam and a second lower energy charged particle beam, wherein the first lower energy charged particle beam and the second lower energy charged particle beam intercept one another to form a self-interference zone; defocusing the self-interference zone to provide a Fresnel fringe, the Fresnel fringe forming an image of the object.

### FIELD

The present technology is an imaging system that employees a reflective advanced focusing lens for focusing charged particles. More specifically, it is a system that can be used to for treating patients, imaging tissue anomalies in patients, and imaging objects.

### BACKGROUND

There are many imaging systems that can be used to image and/or treat patients or image objects. Many are based on acoustics, such as ultrasound or light such as lasers, both of which can be reflected with mirrors and lenses to focus an acoustic beam or a light beam. Charged particles have been used in imaging and/or in treating patients or imaging objects. Examples of imaging objects with charged particles include transmission electron microscopy and scanning electron microscopy. These microscopes include electrostatic beam deflectors, see for example, United States Patent Application Publication No. 20190096630, which discloses a device for, in combination with a stop having an aperture, generating charged particle beam pulses, an apparatus for inspecting a surface of a sample, and a method for inspecting a surface of a sample. The device includes a deflection unit which is arranged for positioning in or along a trajectory of a charged particle beam. The deflection unit is arranged for generating an electric field for deflecting said charged particle beam over the stop and across the aperture. The device also includes an electrical driving circuit for providing a periodic signal. The electrical driving circuit is connected to the manipulation unit via a photoconductive switch, wherein the photoconductive switch is arranged for substantially insulating the deflection unit from the electrical driving circuit, and for conductively connecting the deflection unit to the electrical driving circuit only when said photoconductive switch is illuminated by a light beam. This cannot focus a beam to a three-dimensional focal point more than about 10 centimeters from the source of the charged particle beam.

United States Patent Application Publication No. 20180254168 discloses a method of operating a charged particle microscope comprising the following steps: Providing a specimen on a specimen holder; Using a source to produce a beam of charged particles; Passing said beam through an illuminator comprising: A source lens, with an associated particle-optical axis; A condenser aperture, which is disposed between the source lens and specimen and is configured to define a footprint of said beam upon the specimen; Irradiating the specimen with the beam emerging from said illuminator; Using a detector to detect radiation emanating from the specimen in response to said irradiation, and producing an associated image, specifically comprising the following steps: Choosing a set of emission angles from said source; For each emission angle in said set, selecting a corresponding sub-beam that emits from the source at that emission angle, and storing a test image formed by that sub-beam, thereby compiling a set of test images corresponding to said set of emission angles; Analyzing said set of test images to evaluate illuminator aberrations generated prior to said condenser aperture. This cannot focus a beam to a three-dimensional focal point more than about 10 centimeters from the source of the charged particle beam.

United States Patent Application Publication No. 20180033586 discloses an apparatus and method for exposing a sample. The apparatus comprises a source for electromagnetic radiation or particles having energy, an exposing unit for exposing said sample to said electromagnetic radiation or particles, and a substrate holding device for holding said sample at least during said exposing. The exposing unit comprises a component for manipulating and/or blocking at least part of the electromagnetic radiation or charged particles. The component comprises a cooling arrangement which is arranged for substantially maintaining the component at a predetermined first temperature. The substrate holding device comprises a temperature stabilizing arrangement which is arranged to substantially stabilize the temperature of a sample arranged on said substrate holding device. The temperature stabilizing arrangement comprises a phase change material having a phase change at a second temperature, which is at or near the first temperature. This cannot focus a beam to a three-dimensional focal point more than about 10 centimeters from the source of the charged particle beam.

U.S. Pat. No. 8,481,959 discloses systems and methods of an ion implant apparatus include an ion source for producing an ion beam along an incident beam axis. The ion implant apparatus includes a beam deflecting assembly coupled to a rotation mechanism that rotates the beam deflecting assembly about the incident beam axis and deflects the ion beam. At least one wafer holder holds target wafers and the rotation mechanism operates to direct the ion beam at one of the at least one wafer holders which also rotates to maintain a constant implant angle. This cannot focus a beam to a three-dimensional focal point more than about 10 centimeters from the source of the ion beam.

United States Patent Application Publication No. 20210051795 discloses a neutron source, containing a first proton accelerator for producing a first proton beam having a first energy and a first target for producing a first neutron beam, which first target is connected to the first proton accelerator by a first beam trajectory, and at least one first neutron beam channel serving for guiding the protons exiting the first target, characterized by a second proton accelerator for producing a higher, second energy proton beam from the first proton beam, which second proton accelerator is linked to the first proton accelerator by a second proton accelerator, furthermore the first beam trajectory and the second beam trajectory contain a proton beam deflector arranged on a common section, set up to convey the proton beam along the first beam trajectory to the first target in a first operation state, and along the second beam trajectory to the second proton accelerator in a second operation state, and contain a second target for producing a second neutron beam, which second target is linked to the second proton accelerator by a third beam trajectory. In a similar way the neutron source is also conceivable with a third or even more accelerators and targets. The proton beam deflector is disclosed to be an electromagnetic deflector. This cannot focus a beam to a three-dimensional focal point more than about 10 centimeters from the source of the proton beam.

Design Study for Pulsed Proton Beam Generation by Kim et al. (Nuclear Engineering and Technology, Volume 48, Issue 1, February 2016, Pages 189-199) discloses electrostatic deflectors for deflecting proton beams. This cannot focus a beam to a three-dimensional focal point more than about 10 centimeters from the source of the proton beam.

What is needed is a system for imaging objects that are a distance away from the emitter. The system would preferably also be useful for treating patients and imaging tissue anomalies in patients. It would be preferable if the system generated charged particles which were reflected by electrostatically charged reflectors, resulting in a focused three-dimensional beam. It would be preferable if the system could emit the charged particle beam up to a meter from the focused three-dimensional beam. It would be preferable if the system could be applied to many types of imaging systems, including proton beams for treating cancer, to solve many types of imaging problems.

### SUMMARY

The present technology is a system for imaging objects that are a distance away from the emitter. The system is useful for treating patients and imaging tissue anomalies in patients. The system generates charged particles which are reflected by electrostatically charged reflectors, resulting in a focused three-dimensional beam. The system can emit the charged particle beam up to a meter from the focused three-dimensional beam. The system can be applied to many types of imaging systems, including proton beams for treating cancer, to solve many types of imaging problems.

In one embodiment, a method of imaging an object in a first material having a different charge density to the object is provided, the method comprising: focusing a charged particle beam to a virtual charged particle beam source in the first material; moving the virtual charged particle beam source in and around the object to provide at least one charged particle reflected object beam or at least one charged particle refracted object beam and at least one charged particle bypass beam, wherein the charged particle reflected object beam or the charged particle refracted object beam and the charged particle bypass beam intercept one another to form an interference zone; and defocusing the interference zone to provide a Fresnel fringe, the Fresnel fringe forming an image of the object; or focusing the virtual charged particle beam source on the object to provide a first lower energy charged particle beam and a second lower energy charged particle beam, wherein the first lower energy charged particle beam and the second lower energy charged particle beam intercept one another to form a self-interference zone; defocusing the self-interference zone to provide a Fresnel fringe, the Fresnel fringe forming an image of the object.

The method may further comprise a charged particle beam detector detecting the image of the object.

In the method, the charged particle beam is focused with a combination of an electrostatically charged cone-shaped reflector and an electrostatically charged annular reflector.

In the method, a charged particle in the charged particle beam has a charge and the combination may be tuned to the same charge.

In the method, the charge may be between 50 electron volts to 2 Megaelectron volts.

In the method, the charged particle may be an electron.

In the method, the charged particle may be an ion.

In the method, the charged particle may be a proton.

The method may further comprise moving the virtual charged particle beam source in and around the object to provide the charged particle reflected object beam or the charged particle refracted object beam and the charged particle bypass beam.

The method may further comprise focusing the virtual charged particle beam source on the object to provide the first lower energy charged particle beam and the second lower energy charged particle beam.

The method may further comprise comparing a phase of the charged particle reflected object beam or a phase of the charged particle refracted object beam with a phase of the charged particle bypass beam to provide information about the object.

The method may further comprise comparing an amplitude of the charged particle reflected object beam or an amplitude of the charged particle refracted object beam with an amplitude of the charged particle bypass beam to provide information about the object.

The method may further comprise comparing a phase of the first lower energy charged particle beam with a phase of the second lower energy charged particle beam to provide information about the object.

The method may further comprise comparing an amplitude of the first lower energy charged particle beam with an amplitude of the second lower energy charged particle beam to provide information about the object.

In the method, the information from the phase may be the temperature, composition, magnetic field or electrostatic field of the object and the information from the amplitude may be the charge density of the object.

The method may further comprise identifying the object.

In the method, the object may be identified as a tumour or lesion.

In another embodiment, a system for imaging an object in a first material having a different charge density to the object is provided, the system comprising: a charged particle beam source which emits a charged particle beam; a curved cone-shaped electrostatically charged reflector, charged and positioned to reflect the charged particle beam source to provide a first reflected beam; an annular electrostatically charged reflector charged and positioned to reflect the first reflected beam to provide a second reflected beam and to focus the second reflected beam to a three-dimensional probe; a charged particle beam source actuator in mechanical communication with the charged particle beam source; an annular electrostatically charged reflector actuator in mechanical communication with the annular electrostatically charged reflector; a processor in electronic communication with the charged particle beam source actuator; a memory in communication with the processor and having instructions thereon to instruct the processor to move at least one of the charged particle beam source and annular electrostatically charged reflector such that at least one charged particle reflected object beam or at least one charged particle refracted object beam and a charged particle bypass beam intercept one another to form an interference zone or a first lower energy charged particle beam and a second lower energy charged particle beam to intercept one another to form a self-interference zone, the memory further configured to move the charged particle beam source to produce a Fresnel fringe in the interference zone or self interference zone; and an annular charged particle beam detector positioned to image the Fresnel fringe.

In the system, the memory may include instructions for the processor to sharpen the image.

In the system, the annular charged particle beam detector may be a camera.

In the system, the charged particle beam detector may be located off a charged particle beam axis.

In the system, the charged particle beam detector may be located on a charged particle beam axis below the curved cone-shaped electrostatically charged reflector.

In the system, the detector may be a superconducting quantum interference device (SQUID) detector.

In another embodiment, a system for imaging an object is provided, the system comprising:
an apparatus including: a charged particle beam source which emits a charged particle beam; a curved cone-shaped electrostatically charged reflector, charged and positioned to reflect the charged particle beam source to provide a first reflected beam; an annular electrostatically charged reflector charged and positioned to reflect the first reflected beam to provide a second reflected beam and to focus the second reflected beam to a three-dimensional probe; a charged particle beam source actuator in mechanical communication with the charged particle beam source; an annular electrostatically charged reflector actuator in mechanical communication with the annular electrostatically charged reflector; and a charged particle beam detector; and
a computing device including a processor, a user interface and a memory, the processor in electronic communication with the charged particle beam detector, the memory in communication with the processor and having instructions thereon to instruct the processor to display an image on the user interface.

In the system, the memory may include instructions for the processor to sharpen the image.

The system may further comprise a spatial filter in front of the charged particle beam detector.

In the system, the detector may be a superconducting quantum interference device (SQUID) detector.

In yet another embodiment, method of treating a growth, a tumour or a lesion in a first tissue having a different charge density to the growth, tumour or lesion is provided, the method comprising:
providing a system comprising: a charged particle beam source which emits a charged particle beam; a curved cone-shaped electrostatically charged reflector, charged and positioned to reflect the charged particle beam source to provide a first reflected beam; an annular electrostatically charged reflector charged and positioned to reflect the first reflected beam to provide a second reflected beam and to focus the second reflected beam to a three-dimensional probe; and a charged particle beam detector;
focusing a charged particle beam to a virtual three-dimensional probe in the first tissue; moving the virtual charged particle beam source in the first tissue and the growth, tumour or lesion such that an image is created; and
dwelling the virtual three-dimensional probe on the growth, tumour or lesion.

In the method, the charged particle beam source may be a source of protons.

In the method, the curved cone-shaped electrostatically charged reflector and the annular electrostatically charged reflector may be tuned to have a charge which is the same as a charge of a proton from the source of protons.

### FIGURES

FIG. 1 is a schematic of a charged particle beam imaging system of the present technology.

FIG. 2 is a schematic of an alternative embodiment of the charged particle beam imaging system of FIG. 1.

FIG. 3 is a schematic of an alternative embodiment of the charged particle beam imaging system of FIG. 1.

FIG. 4 is a schematic of an alternative embodiment of the charged particle beam imaging system of FIG. 1.

FIG. 5 is a schematic of an alternative embodiment of the charged particle beam imaging system of FIG. 1.

FIG. 6 is a schematic of an alternative embodiment of the charged particle beam imaging system of FIG. 1.

FIG. 7 is a schematic of a SQUID (superconducting quantum interference device)-based detection system.

FIG. 8 is an overview of a charged particle imaging system using Fresnel fringes.

FIG. 9 is a schematic of beams producing an interference zone.

FIG. 10 is a schematic of beams producing a self-interference zone.

FIG. 11A is a side view of an exemplary device; and FIG. 11B is a perspective view of the exemplary device.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range. Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Charged particle—in the context of the present technology a charged particle is an ion, a proton or an electron.

Dark field detector—in the context of the present technology a dark field detector may be a dark field microscope, a scanning transmission electron microscope, a scanning electron microscope or a camera.

Bright field detector—in the context of the present technology, a bright field detector may be a bright field microscope, a scanning transmission electron microscope, a scanning electron microscope or a camera.

Ion emitter—in the context of the present technology, an ion emitter includes hot filament and hollow cathode ion emitters. A source of ions, more commonly monatomic ions can be any element. A commonly used element is gallium. The ion emitter produces an ion beam with a voltage of ˜1 keV to ˜2 MeV and up to GeV for linear accelerator fusion reactors.

Electron emitter—in the context of the present technology, an electron emitter includes an electron gun. The electron gun produces an electron beam with a voltage of ˜50 eV to ˜1.5 MeV.

Proton emitter—in the context of the present technology a proton emitter includes a cyclotron and a tandem accelerator. The cyclotron produces a proton beam with a voltage of ˜50 keV to ˜2 MeV and up to GeV for linear accelerators.

Reflectors—in the context of the present technology, the reflectors are electrostatically charged and reflect charged particle beams. The reflectors can be made of, for example, but not limited to shiny, high charged particle-reflective metal, specifically austenitic stainless steel, molybdenum coated stainless steel for ion beams and brass, preferably copper-beryllium brass. Their electrostatic charge is tuned to either be the same as the charged particle beam voltage or different to the charged particle beams voltage, depending on the application. The angle of reflection of the charged particle beams is directly proportional to the electrostatic charge of the reflector, with reflection being at ninety degrees to the incoming beam if the electrostatic charge of the reflector is the same as the voltage of the charged particle beam. When a charge is put on the surface of the reflector, the charge will take the shape of the reflector's surface. The charge dissipates or decreases as 1/r where r is the distance from the surface. Both the reflectors are three-dimensional so their surface can be rotated 360 degrees. The reflectors are preferably manufactured using focused ion beam technology.

Cone-shaped reflector—in the context of the present technology, a cone-shaped reflector has a concave wall and is equivalent to a curved cone-shaped reflector. The curvature on the cone-shaped reflector and flat second reflector allows the cone to move up and down or forward and backward on the charged particle beam axis enabling the focused three-dimensional probe position to shift forward and backwards.

Distance—in the context of the present technology, the focused probe can be a distance away from the charged particle emitter, for example, in order to image large breasts and the prostate, the emitter and the focused probe are about 10 centimeters apart. For electron microscopes, the distance between the emitter and the focused probe is about 10 cm. For proton treatment of tumors, the distance between the emitter and the focused probe needs to be about 50 cm to 1 meter. The distance between the emitter and the detector is as much as 1 to 2 meters.

### DETAILED DESCRIPTION

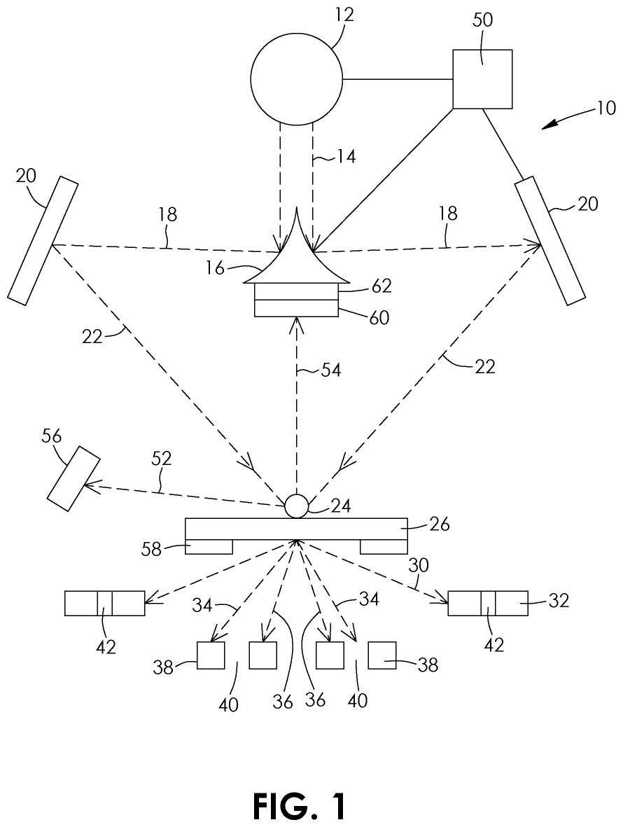

An imaging device, generally referred to as 10 is shown in FIG. 1. A charged particle beam source 12 emits a charged particle beam 14, which strikes a curved cone-shaped electrostatically charged reflector 16. In one embodiment, the cone-shaped electrostatically charged reflector 16 is tuned to have the same charge as the charged particle beam 14. The charged particle beam 14 on the optical axis is reflected at right angles to the incident charged particle beam 14 angle, while those off of the optical axis deviate from right angles. All are referred to as first reflected beams 18 and strike an annular electrostatically charged reflector 20 which is adjacent to the cone-shaped electrostatically charged reflector 16. The annular electrostatically charged reflector 20 is tuned and angled such that the first reflected beams 18 are reflected as second reflected beams 22 at an angle that is equal and opposite to the incident first reflected beam 18 angle and are focused to a small three-dimensional probe (virtual source) 24 which is at the specimen 26 to be imaged or treated. Beams that pass through the specimen 26 are diffracted and are scattered to provide diffusely scattered beams 30. These are detected by an annular dark field detector 32. Other beams pass through the specimen 26 are direct beams 34 and diffracted beams 36. These collectively are referred to as object beams and are detected by an annular bright field detector 38. If an objective aperture is included, the direct beams 34 can be separated from the diffracted beams 36. Both the annular dark field detector 32 and the annular bright field detector 38 include an aperture 40, 42, respectively to allow for the beams to pass through to other detectors. The reflectors 16, 20 are charged with an electrical driving circuit 50 to provide the voltage to the reflectors 16, 20 and the charged particle beam source 12. As the electron beam, the proton beam or the ion beams also lead to diffusely scattered electron beams 52 and backscattered electron beams 54, a diffusely scattered electron beam detector 56 may be placed adjacent to the specimen holder 58, off of the charged particle beam axis and a backscattered electron beam detector 60 may be placed under the support 62 for the cone-shaped electrostatically charged reflector 16 on the charged particle beam axis. The system includes the detectors 32, 38, and optionally, detectors 56, 60.

The direct beam of electrons, protons and ions when passing through a material or object will not deviate much from its original trajectory (only by about 1 degree for the diffracted beams), however, when the beam loses energy by scattering there are many types of signals which can scatter 360 degrees, such as secondary electrons (low energy), backscattered electrons 54 (high energy), x-rays and Auger electrons (very low energy). As a result of the scattering the diffusely scattered electron beam detector 56 placement is highly flexible. As a result of the backscattering, the backscattered electron beam detector 60 may be placed under the cone-shaped electrostatically charged reflector 16 on the emission surface facing the virtual source 24 and on the charged particle beam axis.

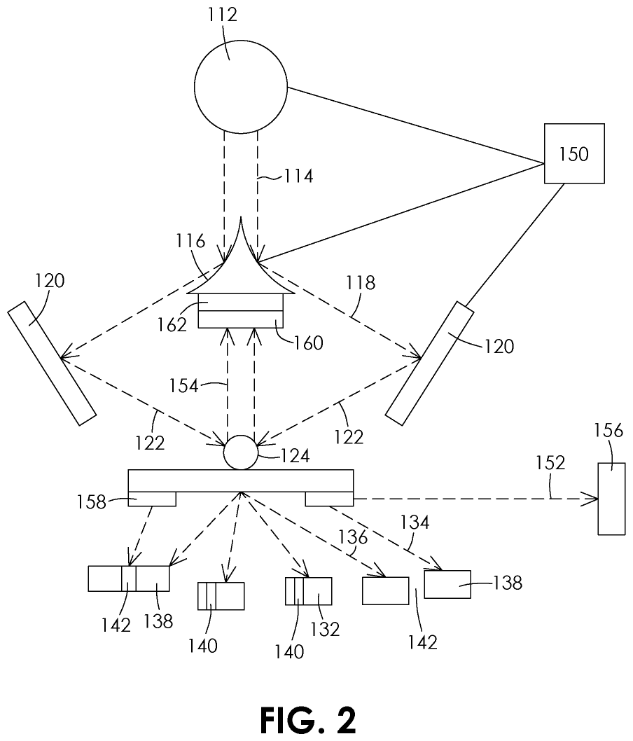

In another embodiment shown in FIG. 2, the curved cone-shaped electrostatically charged reflector 116 is tuned to have a charge that is different to the charged particle beam 114 from the charged particle beam source 112, resulting in the path of the first reflected beams 118 being other than normal to the incident charged particle beam 114.

The annular electrostatically charged reflector 120 is below the cone-shaped electrostatically charged reflector 116. The annular electrostatically charged reflector 120 is positioned such that the first reflected beams 118 strike the annular electrostatically charged reflector 120. The annular electrostatically charged reflector 120 is tuned and angled such that the first reflected beams 118 are reflected as second reflected beams 122 and are focused to a small three-dimensional probe 124 which is at the specimen 126 to be imaged or treated. The remainder of the path is as described in relation to FIG. 1: Beams that pass through the specimen 126 are diffracted and are scattered to provide diffusely scattered beams 130. These are detected by an annular dark field detector 132. Other beams pass through the specimen 126 are direct beams 134 and diffracted beams 136. These collectively are referred to as object beams and are detected by an annular bright field detector 138. Both the annular dark field detector 132 and the annular bright field detector 138 include an aperture 140, 142, respectively to allow for the beams to pass through to other detectors. The reflectors 116, 120 and the charged particle beam source 112 are charged with an electrical driving circuit 150 to provide the voltage to the reflectors 116, 120. As the electron beam, the proton beam or the ion beams lead to diffusely scattered electron beams 152 and backscattered electron beams 154, a diffusely scattered electron beam detector 156 may be placed adjacent to the specimen holder 158, off the charged particle beam axis and a backscattered electron beam detector 160 may be placed under the support 162 for the cone-shaped electrostatically charged reflector 116 on the charged particle beam axis. The system includes the detectors 132, 138, and optionally, detectors 156, 160.

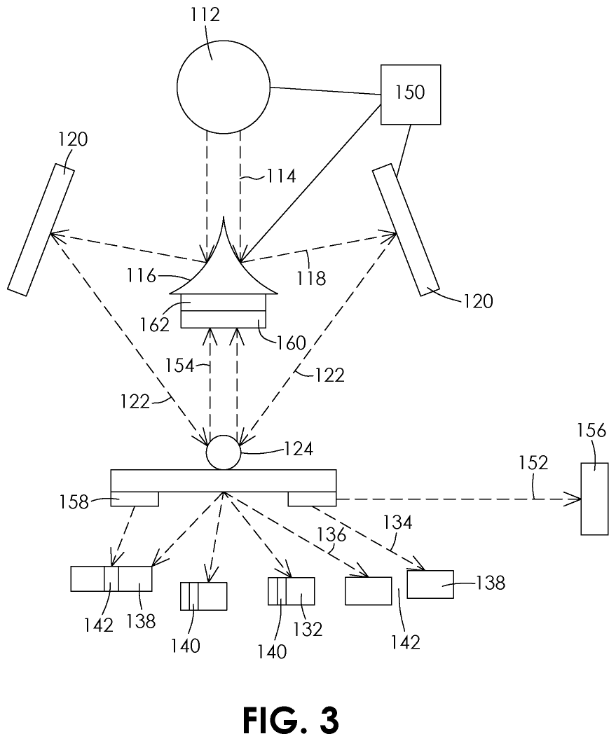

In FIG. 3 the curved cone-shaped electrostatically charged reflector 116 is tuned to have a charge that is different to the charged particle beam 114 from the charged particle beam source 112, resulting in the path of the first defected beams 118 being other than normal to the incident charged particle beam 114. The annular electrostatically charged reflector 120 is positioned such that the first reflected beams 118 strike the annular electrostatically charged reflector 120, in this case above the cone-shaped electrostatically charged reflector 116. The annular electrostatically charged reflector 120 is tuned and angled such that the first reflected beams 118 are reflected as second reflected beams 122 and are focused to a small three-dimensional probe 124 which is at the specimen 126 to be imaged or treated. The annular electrostatically charged reflector 120 is tuned and angled such that the first reflected beams 118 are reflected as second reflected beams 122 and are focused to a small three-dimensional probe 124 which is at the specimen 126 to be imaged or treated. The remainder of the path is as described in relation to FIG. 1: Beams that pass through the specimen 126 are diffracted and are scattered to provide diffusely scattered beams 130. These are detected by an annular dark field detector 132. Other beams pass through the specimen 126 are direct beams 134 and diffracted beams 136. These collectively are referred to as object beams and are detected by an annular bright field detector 138. Both the annular dark field detector 132 and the annular bright field detector 138 include an aperture 140, 142, respectively to allow for the beams to pass through to other detectors. The reflectors 116, 120 and the charged particle beam source 112 are charged with an electrical driving circuit 150 to provide the voltage to the reflectors 116, 120. As the electron beam, the proton beam or the ion beams lead to diffusely scattered electron beams 152 and backscattered electron beams 154, a diffusely scattered electron beam detector 156 may be placed adjacent to the specimen holder 158, off of the charged particle beam axis and a backscattered electron beam detector 160 may be placed under the support 162 for the cone-shaped electrostatically charged reflector 116, in line with the charged particle beam axis. The system includes the detectors 132, 138, and optionally, detectors 156, 160.

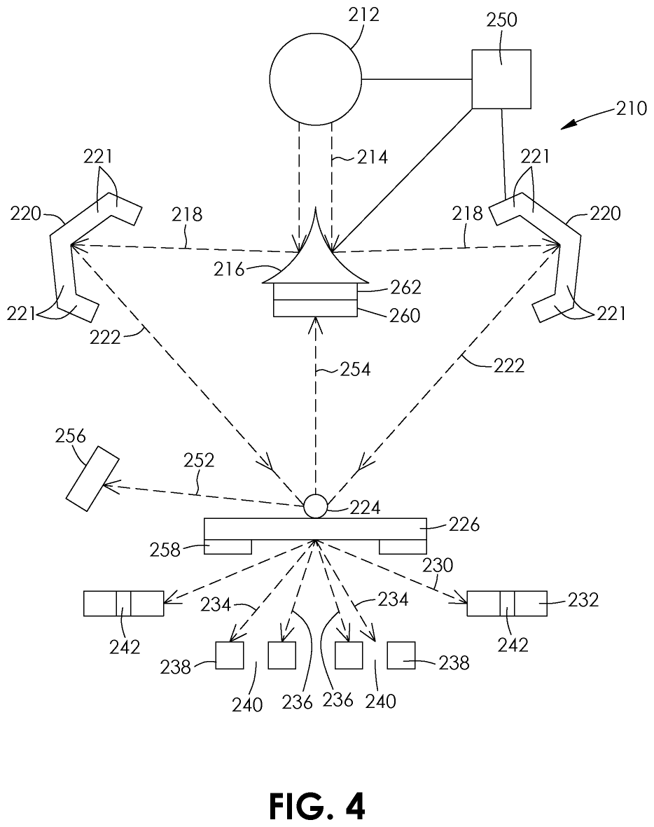

In another embodiment shown in FIG. 4, the curved cone-shaped electrostatically charged reflector 216 is tuned to have a charge that is different to the charged particle beam 214 from the charged particle beam source 212 resulting in the path of the first defected beams 218 being other than opposite and equal to the charged particle beam 214, or it is tuned to have a charge that is the same as the charged particle beam 214. The annular charged reflector 220 is positioned such that the first reflected beams 218 strike the annular electrostatically charged reflector 220. The annular electrostatically charged reflector 220 is segmented into quadrants 221, for example, four, or six or eight. Each quadrant 221 can be individually tuned for its electrostatic charge enabling the second reflected beams 222 and therefore the resultant three-dimensional probe 224 to be shifted off the charged particle beam axis, which enables the second reflected beams 222 to be rastered back and forth, sideway or rotated on the specimen 226 to be treated or imaged. The remainder of the path is as described in relation to FIG. 1: Beams that pass through the specimen 226 are diffracted and are scattered to provide diffusely scattered beams 230. These are detected by an annular dark field detector 232. Other beams pass through the specimen 226 are direct beams 234 and diffracted beams 236. These collectively are referred to as object beams and are detected by an annular bright field detector 238. Both the annular dark field detector 232 and the annular bright field detector 238 include an aperture 240, 242, respectively to allow for the beams to pass through to other detectors. The reflectors 216, 220 and the charged particle beams source 212 are charged with an electrical driving circuit 250 to provide the voltage to the reflectors 216, 220. As the electron beam, the proton beam or the ion beams lead to diffusely scattered electron beams 252 and backscattered electron beams 254, a diffusely scattered electron beam detector 256 may be placed adjacent to the specimen holder 258 and a backscattered electron beam detector 260 may be placed under the support 262 for the cone-shaped electrostatically charged reflector 216. The system includes the detectors 232, 238, and optionally, detectors 256, 260.

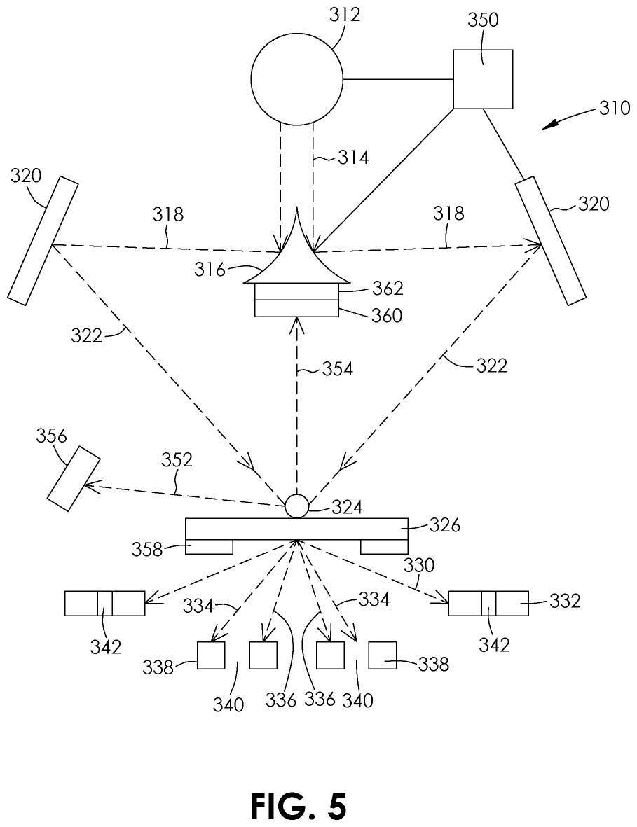

In another embodiment shown in FIG. 5, an electron beam emitter 312, which may be a field emission electron gun or a thermo-ionic electron gun, emits an electron beam 314 which strikes a curved cone-shaped electrostatically charged reflector 316. In one embodiment, the cone-shaped electrostatically charged reflector 316 is tuned to have the same charge as the electron beam 314. In another embodiment, the cone-shaped electrostatically charged reflector 316 is tuned to have a charge that is different to the charged particle beam 314 resulting in the path of the first defected beams 318 being other than opposite and equal to the charged particle beam 314 path. The electron beam 314 is reflected as first reflected beams 318 and strikes an annular electrostatically charged reflector 320 which is adjacent to the cone-shaped electrostatically charged reflector 316. The annular electrostatically charged reflector 320 is tuned and angled such that the first reflected beams 318 are reflected as second reflected beams 322 and are focused to a small three-dimensional probe 324 which is at the specimen 326 to be imaged or treated. Beams that pass through the specimen 326 are diffracted and are scattered to provide diffusely scattered beams 330. These are detected by an annular dark field detector 332. Other beams pass through the specimen 326 are direct beams 334 and diffracted beams 336. These collectively are referred to as object beams and are detected by an annular bright field detector 338. Both the annular dark field detector 332 and the annular bright field detector 338 include an aperture 340, 342, respectively to allow for the beams to pass through to other detectors. The reflectors 316, 320 and the electron beam source 312 are charged with an electrical driving circuit 350 to provide the voltage to the reflectors 316, 320. As the electron beam leads to diffusely scattered electron beams 352 and backscattered electron beams 354, a diffusely scattered electron beam detector 356 may be placed adjacent to the specimen holder 358 and a backscattered electron beam detector 360 may be placed under the support 362 for the cone-shaped electrostatically charged reflector 316. The system includes the detectors 332, 338, and optionally, detectors 356, 360.

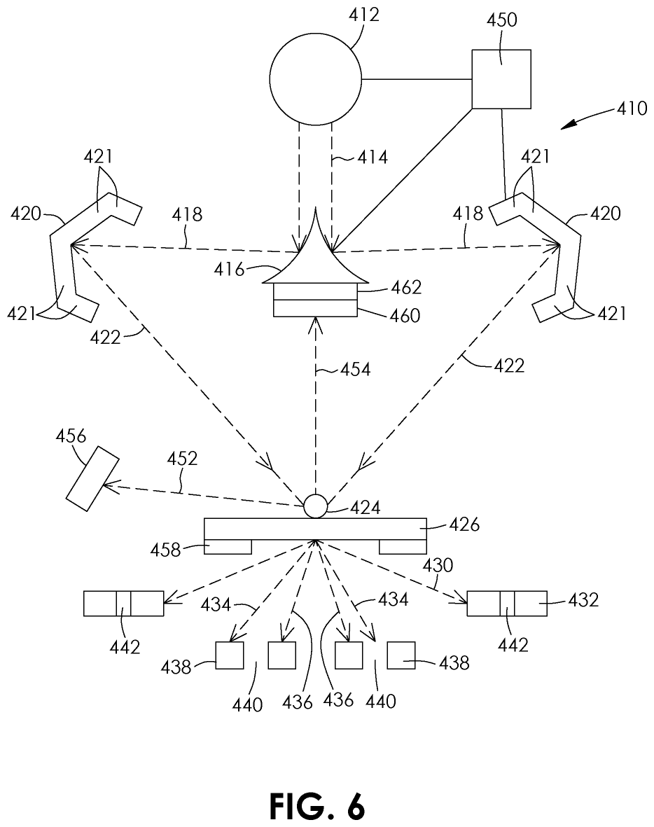

In another embodiment shown in FIG. 6 the curved cone-shaped electrostatically charged reflector 416 is tuned to have a charge that is different to the electron beam 414 resulting in the path of the first defected beams 418 being other than normal to the electron beam 414, or it is tuned to have a charge that is the same as the electron beam 414. The annular electrostatically charged reflectors 420 is positioned such that the first reflected beams 418 strike the annular electrostatically charged reflector 420. The annular electrostatically charged reflector 420 is segmented into quadrants 421, for example, four, or six or eight. Each quadrant 421 can be individually tuned for its electrostatic charge enabling the second reflected beams 422 and therefore the resultant three-dimensional probe (virtual source) 424 to be shifted off the charged particle beam axis, which enables the second reflected beams 422 to be rastered back and forth, sideway or rotated on the specimen 426 to be treated or imaged. The remainder of the path is as described in relation to FIG. 5: Beams that pass through the specimen 426 are diffracted and are scattered to provide diffusely scattered beams 430. These are detected by an annular dark field detector 432. Other beams pass through the specimen 426 are direct beams 434 and diffracted beams 436. These collectively are referred to as object beams and are detected by an annular bright field detector 438. Both the annular dark field detector 432 and the annular bright field detector 438 include an aperture 440, 442, respectively to allow for the beams to pass through to other detectors. The reflectors 416, 420 and the electron beam source 412 are charged with an electrical driving circuit 450 to provide the voltage to the reflectors 416, 420. As the electron beam leads to diffusely scattered electron beams 452 and backscattered electron beams 454, a diffusely scattered electron beam detector 456 may be placed adjacent to the specimen holder 458 and a backscattered electron beam detector 460 may be placed under the support 462 for the cone-shaped electrostatically charged reflector 416. The system includes the detectors 432, 438, and optionally, detectors 456, 460.

In all the embodiments, the detector may be a SQUID detector 500 (superconducting quantum interference device) detector 500, which precisely measures magnetic flux at very high speed. The SQUID detector 500 allows for measuring the phase, the refractive index (mean inner potential) of the specimen, which is the electrostatic potential of the atoms making up a solid material. The SQUID detector 500 may be made of a single SQUID, a linear array of SQUIDs or a two-dimensional array of SQUIDs.

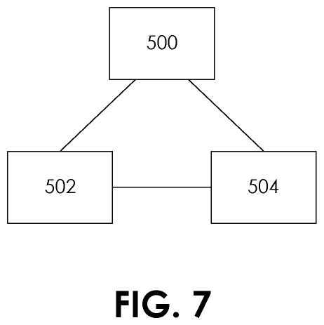

As shown in FIG. 7, the SQUID detector 500 is in electronic communication with a vector network analyzer 502 and a clock 504, for example, but not limited to a rubidium clock. The magnetic flux changes due to the absorption or disturbance of a charge from the electron, proton or ion. The change in magnetic flux in the SQUID detector 500 creates a charge of its own in the SQUID detector 500. Using the vector network analyzer 502 and the rubidium clock 504, the SQUID detector 500 can be used to measure the flight time or time taken from the emitter 12, 112, 212, 312 to the SQUID detector 500. The rubidium clock 504 has a precision in measuring of ˜10exp(−12) seconds, and there's even better, faster clocks than Rubidium. As mentioned, the time it takes for an electron in an electron microscope to go from the emitter to detector (and only one electron is emitted at a time) is a nanosecond. Knowing the path length traveled by the electron, proton or ion and the measured time it takes to pass through the microscope including through the specimen and/or those charges created by the specimen (inelastically scattered secondary, backscattered and Auger electrons) enables the speed of the charged signal to be measured at each pixel in the image of the specimen and to create a phase image. The difference in time from pixel to pixel in the phase image measures the differences in refractive index of the specimen that is due to differences in composition, temperature, strain, electrostatic and magnetic fields within the specimen.

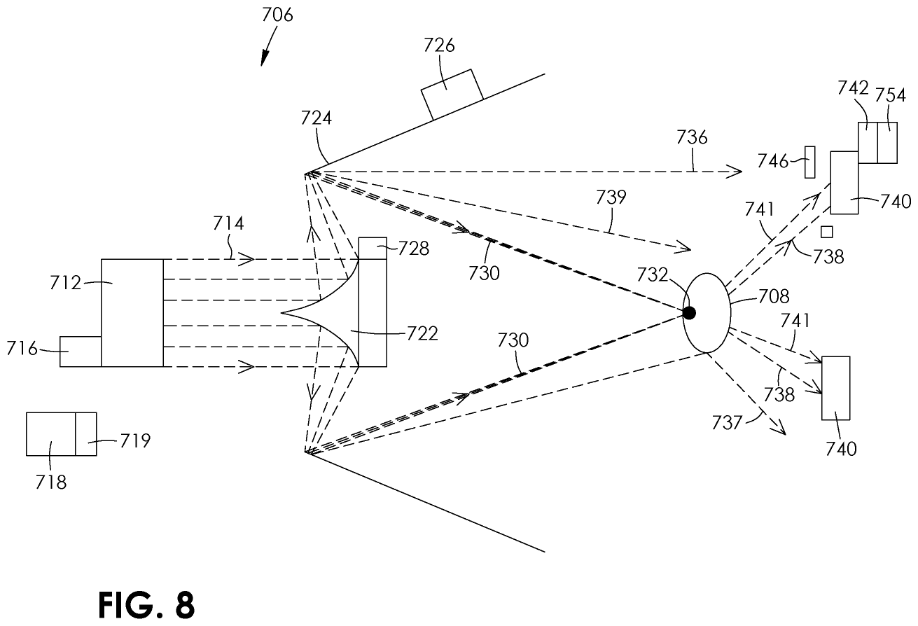

An overview of a charged particle imaging system, generally referred to as 706, for imaging an object, a specimen, a tissue, an organ, or a body part (an object), generally referred to as 708, is shown schematically in FIG. 8. A charged particle source 712 emits a single charged particle beam 714. The charged particle source 712 can be manually moved or can be moved with a source actuator 716 that is in mechanical communication with the charged particle source 712. The source actuator 716 is preferably controlled by a processor 718, which is under control of a memory 719, which has instructions thereon for instructing the processor 718 to actuate the source actuator 716. The charged particle source 712 provides a charged particle beam 714 with a beam voltage between and including ˜50 eV to ˜1.5 MeV for an electron beam, ˜50 keV to ˜2 MeV for a proton beam and ˜50 keV to ˜2 MeV for an ion beam. It can be used for obtaining information including one or more of density, temperature, composition, elasticity, or strain field in a mammalian body.

The charged particle beam 714 has a large cross-sectional area, typically on the order of a centimeter or a few centimeters. The charged particle beam 714 is directed to a curved cone-shaped electrostatically charged reflector 722 and then to an annular electrostatically charged reflector 724 where it is reflected by and focused into a convergent beam 730 that terminates at the object 708 as a virtual source 732. The annular electrostatically charged reflector 724 pivots under control of an annular electrostatically charged reflector actuator 726, which is under control of the processor 718, which in turn is controlled by the memory 719, which has instructions thereon for instructing the processor 718 to actuate the actuator 726. The cone-shaped electrostatically charged reflector 722 is under control of an actuator 728 that moves it towards and away from the charged particle emitter (source) 712. The actuator 728 which is under control of the processor 718, which in turn is controlled by the memory 719, which has instructions thereon for instructing the processor 718 to actuate the actuator 728. The convergent beam 730 converges to a point which is a virtual focused charged particle imaging source 732 at the point of cross-over. The processor 718 under control of the memory 719 is configured to direct the source actuator 716 to cause the charged particle source 712 to move the virtual source 732 into the object 708 and to move it around within the object 708. Further, the processor 718 under control of the memory 719 is configured to move the cone-shaped electrostatically charged reflector 722 towards and away from the annular electrostatically charged reflector 724, thus moving the virtual source 732 towards and away from the charged particle emitter 712, again positioning the virtual source 732 in the object 708. The foregoing components are provided in a device.

The virtual source 732 is positioned inside the object 708. The virtual source 732 transmits a plurality of beams 736 that are scattered by the object in all directions three-dimensionally. By moving the source 732 in the object 708, the virtual source 732 scans the object 708. The virtual source 732 enters into any object 708 that it encounters, then out of the object 708 as direct object beams 738, which are detected by a detector 740. The detector 740 is aimed at the virtual source 732 such that it can detect the direct object beams 738. Some of the direct object beams 738 pass directly through the object 708 while other direct object beams 738 are diffracted as they pass through the object 708. The detector 740 can move to collect direct object beams 738 having a range of angular directions.

The virtual source 732 can be moved around to hit the object 708 from many spots, distances and angles. The virtual source 732 does one of hit the edge of the object and reflect off the edge to produce reflected object beams 737, pass through the object 708 to produce direct object beams 738 and refracted object beams 741 or miss the object 708 to produce bypass beams 739. The bypass beams 739 overlap with the reflected object beams 737.

The detector 740 moves towards and away from the object 708 in order to defocus the image created by combinations of reflected object beams 737 and bypass beams 739, such that it becomes photographically visible. A detector actuator 742 is in mechanical communication with the detector 740 and is under control of the processor 718 that is in electronic communication with the detector actuator 742. Again, the processor 718 receives instructions from the memory 719. The direct object beams 738 also contain information about the object 710. The information carried by the direct object beams 738 is analyzed to determine its amplitude and phase according to techniques known in the art. The phase information of the direct object beam 738 provides information on the object's temperature, composition, magnetic field or electrostatic field and amplitude measurements provide information on the opaqueness or density of the object. A spatial filter 746 reduces the noise from any unwanted scattered beams 736 and is located in front of the detector 740.

The charged particle beams are generated in a vacuum. The proton beam passes through a thin membrane into the ambient environment, and then into the patient being treated. In order for the object 708 to be observed, the virtual source 732 is moved inside the patient by pivoting the annular reflector 724 and the detector 740, or by shifting the device 706, or by repositioning the patient. A vector network analyzer is not required as the amplitude and phase information of the emitted and received intensities is not used to produce the image. However, a better intensity image can result using the vector network analyzer for the temporal filter 754. An intensity image using Fresnel fringes will form without using the temporal filter and spatial filter but using these filters the intensity image will improve, i.e., better spatial resolution, by being able to reduce the apparent size of the virtual source.

For phase or speed of charged particle beam imaging, the vector network analyzer is needed to measure the time difference for receiving the direct object beam 738 at each element in the detector. Since the path length traveled from the charged particle source 712 to the detector 740, by measuring the time using the vector network analyzer, the speed (m/s) can be determined.

The direct object beams 738 can be considered for diagnostic purposes of the object 708. To form a speed of charged particle image using temporal coherence, the emission time of the beam is measured and then the arrival time of the beam at each pixel in the image is measured. Any differences in the speed of the charged particle beam across the image can be used to diagnose structures in the image.

The spatial interference between the reflected object beams 737 and the bypass beams 739 in the overlap is used to create the image. The image of the object 708 can be considered as an inline hologram for diagnostic purposes of the object 708. More specifically, the image is created using the principle of Fresnel diffraction.

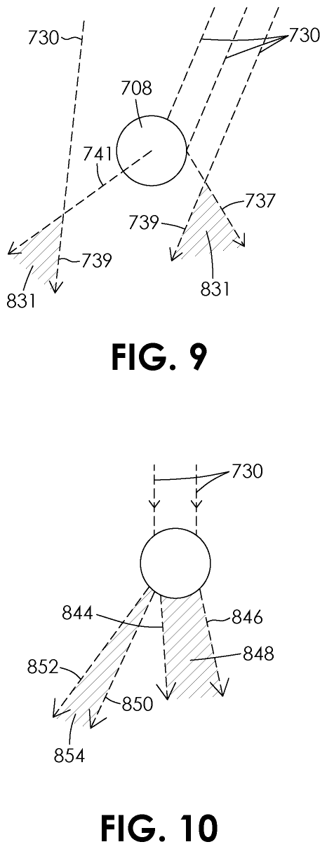

As shown in FIG. 9, the bypass beam 739 overlaps with the reflected object beam 737 to produce an interference zone 841. This interference zone 841 between these two beams forms the Fresnel fringe or defocus fringe. Some intensity in the defocus or Fresnel fringe also comes from the refracted object beams 741 (diffracted beams 436) exiting the object 708 and interfering with the bypass beam 739 in the same manner in which the bypass beam and the reflected object beam 737 interfere although the refracted object beam 741 has a less significant role in the image formation process of the Fresnel fringe. As shown in FIG. 10, yet another way that an image is produced is for direct beams 842 (direct beams 434) that lose energy to provide at least a first lower energy direct beam 844 (first lower energy object beam) and a second lower energy direct beam 846 (second lower energy object beam) to interfere with each other to provide a self-interference zone 848. This self-interference zone 848 forms the Fresnel fringe or defocus fringe, which creates the image. Yet another way that an image is produced is for refracted object beams 741 that lose energy to provide at least a first lower energy refracted object beam 850 and a second lower energy direct beam 852 (second lower energy object beam) to interfere with each other to provide a self-interference zone 854. This self-interference zone 854 forms the Fresnel fringe or defocus fringe, which creates the image.

The Fresnel diffraction produces a fringe (Fresnel fringe) in an image when defocusing occurs. The Fresnel fringe enhances the contrast between the forms and the background and allows for the imaging of soft tissues, and the interface between different soft tissues. This includes tissues that have very little difference in charge density, such as for example, but not limited to, breast tissue and milk glands in the breast tissue, lesions in tissues, and an egg in a fallopian tube.

The width of the overlap increases with defocus, which increases the width of the Fresnel fringe. The defocus decreases to zero where the object and camera are on the same plane. In this condition, the object disappears and can't be seen because a fringe cannot be made as there is no overlap.

Additionally, the spatial resolution is determined by the width of the Fresnel fringe. The smallest width of the Fresnel fringe found in the image is the size of the virtual source size. The size of the virtual source is determined by the focusing ability of the device 735 and the wavelength of the emitted charged particle beam from the emitter. For example, the wavelength at 100 keV, 200 keV, and 300 keV is 3.70 picometers (pm), 2.51 pm and 1.96 pm. This is much higher resolution than acoustic or light beams.

An image formed with a large defocus, i.e., broad fringe lines, can be processed with the processor to sharpen the features (i.e., the Fresnel fringes) in the image by applying a defocus amount, delta f, and knowing the cone angle, alpha of the beam such that the reduction in fringe width is delta f times ½ the cone angle. Likewise, knowing the cone angle and the change in fringe width by a known or measured defocus can be used to determine the distance or position of the object, z, in the image, enabling a 3D image to be produced since the lateral dimensions, x, y, are already measurable in the image.

The distance between the virtual source 732 and the object 708 determines the magnification of the object. The further the virtual source 732 is to the object 708 the closer the magnification approaches one. The magnification of the object increases the closer the virtual source 732 approaches the object 708.

The spatial filter 746 and a temporal filter 754 (see FIG. 8), which is provided by the software in the memory 719, restrict the volume of the charged particle virtual source 732 used for imaging, with the smaller the volume, the better the resolution for imaging. The spatial resolution, in part, is set by the size of the convergent beam 730 at the focused virtual source 732. The spatial filter 746 defines a lateral x,y dimension or angular acceptance angle of the virtual source 732. The spatial filter 746 can be made smaller than the virtual source 34 and is one of the factors that determines the spatial resolution. It can be the determining factor that determines the spatial resolution in the x,y plane. The temporal filter 754 determines the z or axial spatial resolution of the virtual source 732. It determines the acceptance time for receiving the charged particle signal start time and object beam stop time.

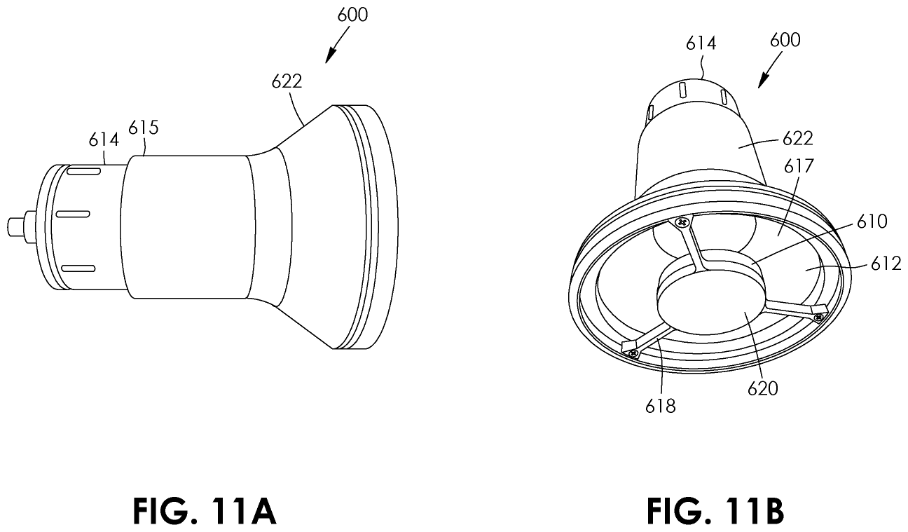

An exemplary device is shown in FIGS. 11A and 11B. Although the elements are shown as for FIG. 1, the same design is suitable for FIGS. 1-4. As shown in FIG. 11A, the charged particle emitter 614 is at one end 615 of the housing 622. As shown in FIG. 11B, the concave, curved cone-shaped reflector 610 is located in the interior 617 of the housing 622 and is retained by support members 618. The annular reflector 612 is on the inside of the housing 622. The charged particle detector 620 lies on the outer surface of the concave, curved, cone-shaped detector. The device 600 may be hand-held and user adjusted and actuated, or it may be under control of a processor and memory in a computing device as described above. Regardless, the computing device includes a user interface on which the image is displayed.

The device and systems can be applied to many types of imaging systems to solve many types of imaging problems including proton beams treating cancer because the tissue before and after the tumor can be damaged much less. For treating a growth, tumour or lesion, the device and system first image the growth, tumour or lesion and then treat the growth, tumour or lesion by dwelling the three-dimensional probe on the growth tumour or lesion at a dwell time that would be known to one skilled in the art. The device or systems described above can be used to produce images based on backscattered beams, based on diffusely scattered beams, and based on Fresnel fringes.

The imaging device 10 can correct for all of the focusing aberrations of the beam used to form the far distant, high-intensity (100% of the intensity), 3D probe used for medical treatment and imaging purposes. There are three main aberrations, spherical aberration, coma and chromatic aberration. The first two, spherical and coma, are corrected by focusing the probe onto the optic axis of the microscope. If the beam is spherically aberrated when traveling on one side of the optic axis, i.e., off the optic axis, then when it is reflected back to the optic axis, there is an additional spherical aberration to the beam that is equal and opposite of the initial spherical aberration, which results in the cancelation of the spherical aberration. Same with coma. Chromatic aberration is a little more involved. It's caused by acceleration voltage fluctuations of the beam. It is compensated by the RAFA lens's (reflective advanced focusing aperture lens) (the device 10) surfaces, i.e., cone-shaped lens and reflective mirror, being “soft” electromagnetically, i.e., the reflective potential of the surfaces becomes equal and opposite to the acceleration voltage fluctuations of the microscope canceling the chromatic aberration. For example, if the acceleration voltage is 100,000 eV+−1 eV, then when the acceleration voltage reduces to 99,999 eV the surface of the RAFA lens also reduces to 99,999 eV. This maintains the reflection angle of the beam off the surfaces of the RAFA lens thus correcting the chromatic aberration. The acceleration voltage of the microscope is controlled by the high-tension tank. Both the microscope's acceleration tubes and the RAFA lens can be tied into the high-tension tank for them to have equal voltages.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A method of imaging an object in a first material having a different charge density to the object, the method comprising: focusing a charged particle beam to a virtual charged particle beam source in the first material; moving the virtual charged particle beam source in and around the object to provide at least one charged particle reflected object beam or at least one charged particle refracted object beam and at least one charged particle bypass beam, wherein the charged particle reflected object beam or the charged particle refracted object beam and the charged particle bypass beam intercept one another to form an interference zone; and defocusing the interference zone to provide a Fresnel fringe, the Fresnel fringe forming an image of the object; or focusing the virtual charged particle beam source on the object to provide a first lower energy charged particle beam and a second lower energy charged particle beam, wherein the first lower energy charged particle beam and the second lower energy charged particle beam intercept one another to form a self-interference zone; defocusing the self-interference zone to provide a Fresnel fringe, the Fresnel fringe forming an image of the object.

2. The method of claim 1, further comprising a charged particle beam detector detecting the image of the object.

3. The method of claim 2, wherein the charged particle beam is focused with a combination of an electrostatically charged cone-shaped reflector and an electrostatically charged annular reflector.

4. The method of claim 3, wherein a charged particle in the charged particle beam has a charge and the combination is tuned to the same charge.

5. The method of claim 4, wherein the charge is between 50 electron volts to 2 Megaelectron volts.

6. (canceled)

7. (canceled)

8. The method of claim 5, wherein the charged particle is a proton.

9. The method of claim 8, further comprising moving the virtual charged particle beam source in and around the object to provide the charged particle reflected object beam or the charged particle refracted object beam and the charged particle bypass beam.

10. The method of claim 8, further comprising focusing the virtual charged particle beam source on the object to provide the first lower energy charged particle beam and the second lower energy charged particle beam.

11. The method of claim 9, further comprising comparing a phase of the charged particle reflected object beam or a phase of the charged particle refracted object beam with a phase of the charged particle bypass beam to provide information about the object.

12. The method of claim 11, further comprising comparing an amplitude of the charged particle reflected object beam or an amplitude of the charged particle refracted object beam with an amplitude of the charged particle bypass beam to provide information about the object.

13. The method of claim 10, further comprising comparing a phase of the first lower energy charged particle beam with a phase of the second lower energy charged particle beam to provide information about the object.

14. The method of claim 13, further comprising comparing an amplitude of the first lower energy charged particle beam with an amplitude of the second lower energy charged particle beam to provide information about the object.

15. (canceled)

16. (canceled)

17. (canceled)

18. A system for imaging an object in a first material having a different charge density to the object, the system comprising: a charged particle beam source which emits a charged particle beam; a curved cone-shaped electrostatically charged reflector, charged and positioned to reflect the charged particle beam source to provide a first reflected beam; an annular electrostatically charged reflector charged and positioned to reflect the first reflected beam to provide a second reflected beam and to focus the second reflected beam to a three-dimensional probe; a charged particle beam source actuator in mechanical communication with the charged particle beam source; an annular electrostatically charged reflector actuator in mechanical communication with the annular electrostatically charged reflector; a processor in electronic communication with the charged particle beam source actuator; a memory in communication with the processor and having instructions thereon to instruct the processor to move at least one of the charged particle beam source and annular electrostatically charged reflector such that at least one charged particle reflected object beam or at least one charged particle refracted object beam and a charged particle bypass beam intercept one another to form an interference zone or a first lower energy charged particle beam and a second lower energy charged particle beam to intercept one another to form a self-interference zone, the memory further configured to move the charged particle beam source to produce a Fresnel fringe in the interference zone or self interference zone; and an annular charged particle beam detector positioned to image the Fresnel fringe.

19. (canceled)

20. The system of claim 19 wherein the annular charged particle beam detector is a camera.

21. The system of claim 20 wherein the charged particle beam detector is located off a charged particle beam axis.

22. The system of claim 20 wherein the charged particle beam detector is located on a charged particle beam axis below the curved cone-shaped electrostatically charged reflector.

23. The system of claim 18 wherein the detector is a superconducting quantum interference device (SQUID) detector.

24. A system for imaging an object, the system comprising:
an apparatus including: a charged particle beam source which emits a charged particle beam; a curved cone-shaped electrostatically charged reflector, charged and positioned to reflect the charged particle beam source to provide a first reflected beam; an annular electrostatically charged reflector charged and positioned to reflect the first reflected beam to provide a second reflected beam and to focus the second reflected beam to a three-dimensional probe; a charged particle beam source actuator in mechanical communication with the charged particle beam source; an annular electrostatically charged reflector actuator in mechanical communication with the annular electrostatically charged reflector; and a charged particle beam detector; and
a computing device including a processor, a user interface and a memory, the processor in electronic communication with the charged particle beam detector, the memory in communication with the processor and having instructions thereon to instruct the processor to display an image on the user interface.

25. The system of claim 24, wherein the memory includes instructions for the processor to sharpen the image.

26. The system of claim 24, further comprising a spatial filter in front of the charged particle beam detector.

27. (canceled)

28. (canceled)

29. (canceled)

30. (canceled)
